(19) United States
(12) Patent Application Publication
(10) Pub. No.: US 2018/0193638 A1
(43) Pub. Date: Jul. 12, 2018

(54) WEARABLE INJECTION DEVICE INTEGRATING IONTOPHORESIS AND MICRONEEDLE

(71) Applicant: BioFirst Corporation, Taipei (TW)

(72) Inventors: Tsung-Shann JIANG, Taipei (TW); Chang-Jen JIANG, Taipei (TW)

(21) Appl. No.: 15/492,416

(22) Filed: Apr. 20, 2017

(30) Foreign Application Priority Data
Jan. 12, 2017 (TW) 106101006

(51) Int. Cl. A61N 1/30; A61M 37/00; A61N 1/04

(52) U.S. Cl. CPC A61N 1/306; A61M 37/0015; A61M 2210/083; A61M 2209/088; A61M 2037/0023; A61N 1/0428

(57) ABSTRACT

A wearable injection device includes: a driven unit; a microneedle injection patch having an injection therein and located on a side surface of the driven unit; and a wearable loop having two ends connected to the two end sides of the driven unit, respectively; wherein the driven unit has a first electrical property and the wearable loop has a second electrical property at a section opposite the driven unit; wherein the microneedle patch includes a microneedle array connected to the injection and attached on a skin of a biological limb, and the wearable loop surrounds the biological limb, and a direct current resulted from the first electrical property of the driven unit and the second electrical property of the wearable loop leads the injection injected into the biological limb via the microneedle array.

### FIELD OF THE INVENTION

The present invention relates to a wearable device and more specifically to a wearable injection device integrating iontophoresis and microneedle.

### BACKGROUND OF THE INVENTION

Traditionally, subcutaneous injection and oral are two primary treatments for drug administration. Subcutaneous injection can effectively delivery drug, but arising infection and pain. Subcutaneous injection may require much time and cause patient painful during clinical treatment. Accordingly, a treatment of Transdermal Drug Delivery System has rapidly developed, and Microneedle (MN) is a new biomedical injection technology to lower the organization damage of a patient and ease the pain of the patient during the treatment.

Microneedle is manufactured via MEMS to minimize the needles. The Microneedle includes needles having tens of microns diameter and are arranged in a matrix. the tiny needle can penetrate the cuticle of skin without activate the pain nerve of the deep organization, therefore largely decreasing the pain of the patient when taking drug injection in clinical treatment, and the pain-free injection can be realized.

The microneedle includes solid microneedle, hollow microneedle and semi-hollow microneedle. The hollow microneedle is suitable for more precisely control the drug dose, by coordinating with controlling drug release, a long-term drug administration can be achieved; and by in together with Pressurizing injection, the drug can be rapidly transferred. The solid microneedle can be coated with a drug, and the drug can be transferred by spreading when the solid microneedle penetrating into the skin of the patient.

### SUMMARY OF THE INVENTION

A wearable injection device includes: a driven unit; a microneedle injection patch having an injection therein and located on a side surface of the driven unit; and a wearable loop having two ends connected to the two end sides of the driven unit, respectively; wherein the driven unit has a first electrical property and the wearable loop has a second electrical property at a section opposite the driven unit; wherein the microneedle patch includes a microneedle array connected to the injection and attached on the skin of a biological limb, and the wearable loop surrounds the biological limb, and a direct current resulted from the first electrical property of the driven unit and the second electrical property of the wearable loop leads the injection injected into the biological limb via the microneedle array.

To make the aforesaid and other objects, features and advantages of the present invention can be more apparent and easier to be understood, some embodiments are introduced in following quotes, and in together with the accompanying drawings to make a detailed description below.

### BRIEF DESCRIPTION OF THE DRAWINGS

FIG. 1 illustrates a side view diagram of wearable injection device integrating iontophoresis and microneedle of the present invention.

FIGS. 2A and 2B illustrate two schematic diagrams of a microneedle injection patch of wearable injection device integrating iontophoresis and microneedle of the present invention, wherein FIG. 2A is a side cross-sectional diagram, and FIG. 2B is a top view diagram.

FIG. 3 illustrates a schematic diagram of an iontophoresis mechanism of wearable injection device integrating iontophoresis and microneedle of the present invention.

### DETAILED DESCRIPTION OF THE INVENTION

### Definitions

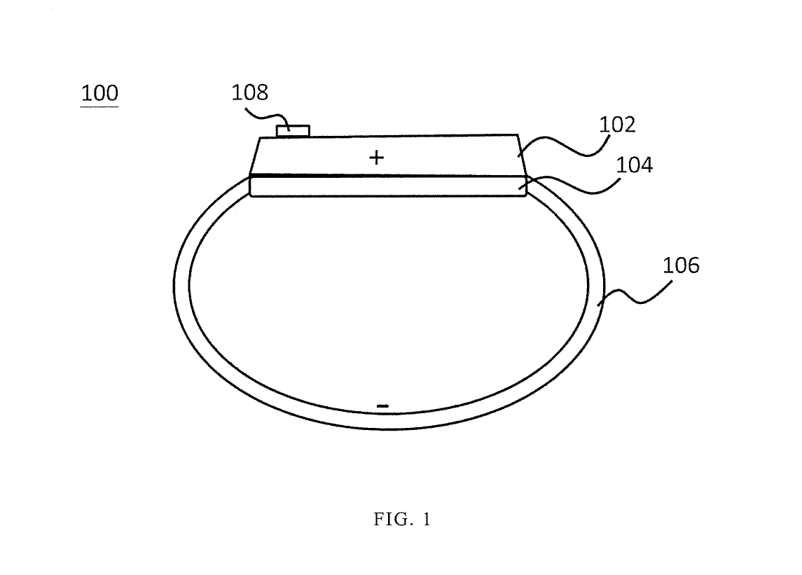

Please refer to FIG. 1, a side view diagram of wearable injection device integrating iontophoresis and microneedle of the present invention is illustrated. The wearable injection device 100 of the present invention includes: a driven unit 102 with a first electrical property; a microneedle injection patch 104 located on a side surface of the driven unit 102; and a wearable loop 106 having two ends connected to the two sides of the driven unit, respectively, and having a second electrical property at a section opposite the driven unit.

In an embodiment, the driven unit 102 can include an electrode (not shown) therein, and the electrode can provide the first electrical property such as positive electricity, and the wearable loop 102 can have the other electrode (not shown) providing the second electrical property such as negative electricity.

In an embodiment, the driven unit 102 can include an activate switch on a surface opposite the microneedle injection patch 104 to start up a direct electrical field between the driven unit 102 and the wearable loop.

In an embodiment, the driven unit 102 can include a battery module (not shown) configured to provide electrical power.

In an embodiment, the material of the wearable loop can include flexible material such as rubber or leather.

In an embodiment, the microneedle patch 104 is connected to the driven unit 102; and in another embodiment, the microneedle injection patch 104 can be detachable from the driven unit, that is, for a driven unit 102, the microneedle patch 104 can be substituted.

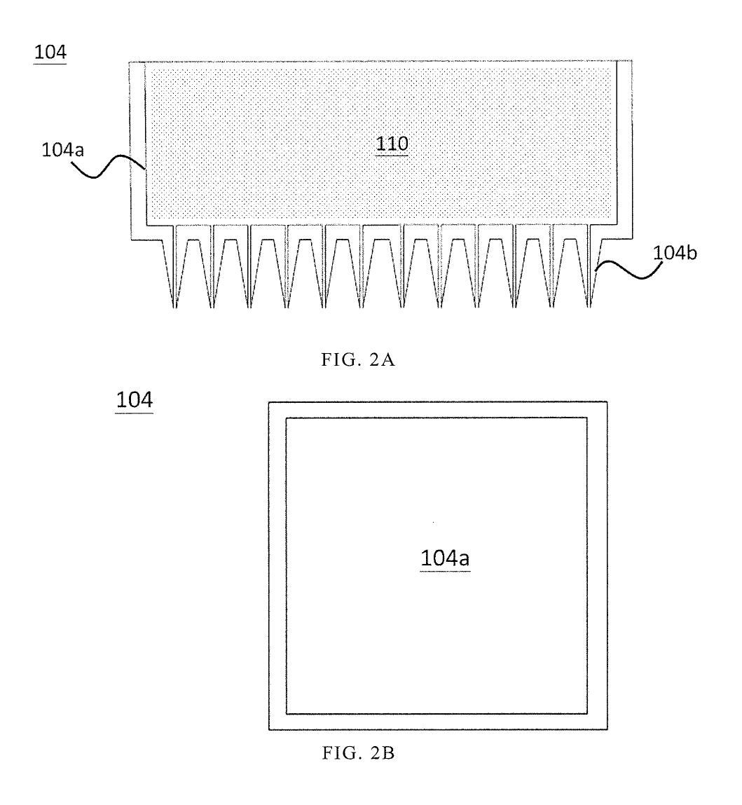

Please refer to FIGS. 2A and 2B, two schematic diagrams of a microneedle injection patch of wearable injection device integrating iontophoresis and microneedle of the present invention are illustrated, and further refer to FIG. 1. The microneedle injection patch 104 can include: a storing tank 104a configured to store an injection 110; and a microneedle array 104b. The storing tank 104a is configured to be in contact with the driven unit 102 and adjacent the electrode in the driven unit 102. The microneedle array 104b includes a plurality of microneedles each having tip directing a direction far away the driven unit 102. Each of the microneedles can be hollow and communicate the storing tank 104a. The injection 110 can include insulin. The material of the microneedle can include SiO₂.

Practically, in an embodiment that the microneedle injection patch 104 is detachable from the driven unit 102, firstly, the microneedle injection patch 104 can be attached on a biological limb such as a skin of a wrist of a human, and a pressure can be slightly loaded on the microneedle injection patch 104 to lead the microneedles penetrating the skin of the biological limb. Then the driven unit 102 can be attached on the microneedle injection patch 104 and at the same time the biological limb can be surrounded by the wearable loop 106. Finally the activate switch is pressed to generate a direct electrical field between the driven unit and the wearable loop, and a direct current is formed in the biological limb, therefore the injection 110 can be ionized to go through the skin of the biological limb via the microneedle array 104b of the microneedle injection patch 104.

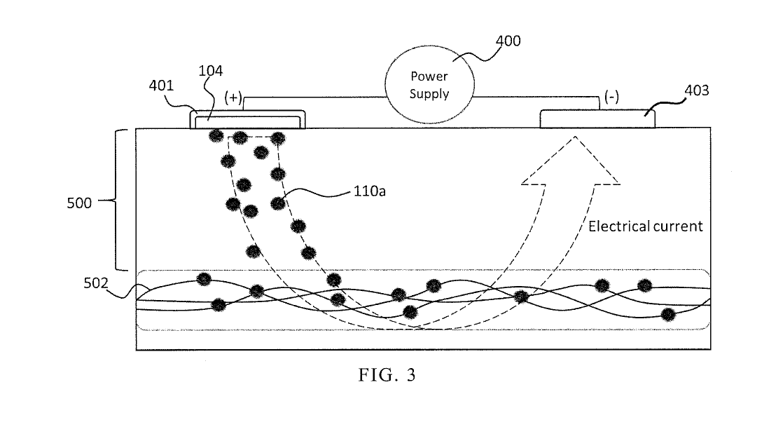

Please refer to FIG. 3, a schematic diagram of an iontophoresis mechanism of wearable injection device integrating iontophoresis and microneedle of the present invention is illustrated. An electrical power supply unit 400 having positive and negative connections connected to a first electrode 401 such as a positive electrode and a second electrode 403 such as a negative electrode. The first electrode 401 can attach a microneedle injection patch thereon, and the microneedle injection patch can have an injection such as insulin. The electrical power supply unit 400 can provide a current from the first electrode 401, passing the skin 500 of the biological limb and flowing to the second electrode 403, and the ionized injection can be carried into the biological limb by the current and absorbed by the vein 502.

In an embodiment, each microneedle has a length between a range from 100 μm to 1500 μm for penetrating cuticle having thickness from 10 μm to 20 μm and can penetrate a depth from 70 μm to 200 μm from the epidermis.

In an embodiment, the driven unit 102 can further include a current adjusting unit (not shown) to adjust the direct current, therefore deciding the speed of the injection entering the biological limb.

In an embodiment, when the injection is insulin, the driven unit 102 can further include a blood glucose detection module (not shown), and the current adjusting unit can automatically control the speed of the insulin entering the biological limb such as a wrist of a human.

The microneedles of the microneedle injection patch can be manufactured by MEM process to produce microneedle array having larger area. The common manufacturing method can include improved LIGA micron manufacturing technology, a composition of RIE and isotropic etching technology, Femtosecond Two Photon Polymerization Technique, DXRL, Photolithography, ICP or FIB.

Conventionally, a macromolecular drug such as insulin is not easily absorbed by the body simply using the microneedle injection patch, however by the wearable injection device of the present invention, a macromolecular drug such as insulin can more easily penetrate the skin to be absorbed via the skin, so that the blood sugar in patients with diabetes can remain stable.

Although the present application has been explained above, it is not the limitation of the range, the sequence in practice, the material in practice, or the method in practice. Any modification or decoration for present application is not detached from the spirit and the range of such.

What is claimed is:

1. A wearable injection device, comprising:
a driven unit;
a micron injection patch having an injection inside and located on a side surface of the driven unit; and
a wearable loop having two sides connected to two sides of the driven unit, respectively;
wherein the driven unit has a first electrical property, and the wearable loop has a second electrical property at a section opposite the driven unit;
wherein the microneedle patch includes a microneedle array connected to the injection and attached on the skin of a biological limb, and the wearable loop surrounds the biological limb, and a direct current resulted from the first electrical property of the driven unit and the second electrical property of the wearable loop leads the injection injected into the biological limb via the microneedle array.

2. The wearable injection device according to claim 1, wherein the driven unit comprises an activate switch to activate a direct current between the driven unit and the wearable loop.

3. The wearable injection device according to claim 1, wherein the first electrical property comprises positive electricity and the second electrical property comprises negative electricity.

4. The wearable injection device according to claim 1, wherein the injection comprises insulin.

5. The wearable injection device according to claim 1, wherein the microneedle injection patch further comprises a storing tank storing the injection.

6. The wearable injection device according to claim 1, wherein the driven unit comprises a current-adjusting unit to adjust the value of the direct current, and deciding the speed of transferring the injection.

7. The wearable injection device according to claim 1, wherein the biological limb comprises a wrist of a human.

8. The wearable injection device according to claim 1, wherein the material of the wearable loop comprises flexible material.

9. The wearable injection device according to claim 1, wherein the driven unit comprise a first electrode providing the first electrical property and the wearable comprises a second electrode providing the second electrical property

10. The wearable injection device according to claim 1, wherein the driven unit comprises an electrode module.

11. The wearable injection device according to claim 1, wherein the microneedle patch is detachable from the driven unit.

12. The wearable injection device according to claim 1, wherein the microneedle patch is attached on the driven unit.
